(19) United States
(12) Patent Application Publication
(10) Pub. No.: US 2006/0155375 A1
(43) Pub. Date: Jul. 13, 2006

(54) Devices and methods for attachment of a gastrointestinal sleeve

(76) Inventors: Jonathan Kagan, Hopkins, MN (US); Mitchell Dann, Wilson, WY (US); Josh Butters, Chandler, AZ (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/236,212

(22) Filed: Sep. 27, 2005

Related U.S. Application Data
(60) Provisional application No. 60/613,917, filed on Sep. 27, 2004.

(51) Int. Cl. A61F 2/02

(52) U.S. Cl. 623/11.11

(57) ABSTRACT

Aspects of this invention disclose devices and methods for attachment of a gastrointestinal sleeve. In some embodiments, the attachment device comprises a flexible cuff, a gastrointestinal sleeve interface, and a gastrointestinal sleeve. Some aspects of the invention contemplate the use of T-tags for attachment of the cuff to tissue. In some aspects of the invention, attachment of the sleeve to the cuff is achieved using various fasteners.

### BACKGROUND OF THE INVENTION

1. Field of the Invention

The present invention relates to devices and methods for treatment of obesity, especially morbid obesity. In particular, the present invention relates to devices and methods for attachment of a gastrointestinal sleeve device within a patient's digestive tract for treatment of obesity.

2. Description of the Related Art

This patent application claims the benefit of U.S. provisional patent application 60/613,917, filed on Sep. 27, 2004, by Kagan et al. for Devices and Methods for Attachment of a Gastrointestinal Sleeve and U.S. utility patent application 11/124,634, filed on May 5, 2005, by Kagan et al. for Devices and Methods for Attaching an Endolumenal Gastrointestinal Implant. These patent applications are hereby expressly incorporated by reference in their entireties herein.

The subject matter of this patent application is related to the following commonly owned and copending patent applications, each of which is hereby incorporated by reference in its entirety, U.S. utility patent application Ser. No. 10/698,148 filed on Oct. 31, 2003 by Kagan et al. for Apparatus and Methods for Treatment of Morbid Obesity and U.S. utility patent application 11/025,364, filed on Dec. 29, 2004, by Kagan et al. for Devices and Methods for Treating Morbid Obesity. The devices and methods described herein can be combined with and/or used in conjunction with the apparatus and methods described in these prior applications.

Gastrointestinal sleeve devices for treatment of obesity have been described in the prior applications listed above, as have various devices and methods for attachment of a gastrointestinal sleeve device within a patient's digestive tract. The present invention is the result of continued investigation into devices and methods for attachment of a gastrointestinal sleeve device within a patient's digestive tract.

### SUMMARY OF THE INVENTION

Aspects of this invention disclose an attachment device comprising a flexible tubular cuff for attachment to tissue, a gastrointestinal sleeve interface, and a gastrointestinal sleeve removably attached to the cuff at the sleeve interface. The cuff, in some embodiments, comprises a plurality of holes for receiving T-tag anchors. The sleeve, in some embodiments, comprises a plurality of holes for attachment to the cuff using fasteners.

### BRIEF DESCRIPTION OF THE DRAWINGS

FIG. 1A illustrates an attachment device with specific functional zones delineated.

FIG. 1B illustrates a sleeve with a rigid ring at its proximal end designed to interface with the rigid ring at the distal end of the attachment cylinder of FIG. 1A.

FIG. 2A shows ingrowth material with holes placed against the gastric mucosa.

FIG. 2B illustrates a tissue ingrowth mesh composite for use in the gastric wall interface zone of an attachment cuff or other implant device.

FIG. 3 illustrates an attachment device with a rigid or semi-rigid sleeve interface attached to the gastric wall by way of flexible isolators to provide attachment compliance.

FIGS. 4A-4B illustrate a basic intra-lumen attachment cuff with an attachment interface for a gastrointestinal sleeve or other device.

FIGS. 5A-5B illustrate an alternate basic intra-lumen attachment cuff with an attachment interface for a gastrointestinal sleeve or other device.

FIGS. 6A-6C illustrate an extra-lumen attachment cuff with an attachment interface for a gastrointestinal sleeve.

FIGS. 7A-7B illustrate an intra-lumen attachment cuff with an attachment interface for a gastrointestinal sleeve or other device.

FIGS. 8A-8B illustrate an intra-lumen attachment cuff with an attachment interface for a gastrointestinal sleeve or other device on the top surface of the attachment cuff.

FIG. 8C illustrates a cross-section of a ring at the top and bottom of the attachment cuff of FIGS. 8A-8B.

FIGS. 9A-9B illustrate an intra-lumen attachment cuff and a gastrointestinal sleeve with an attachment interface for attaching to the cuff.

FIG. 10 illustrates an extra-lumen attachment cuff with an attachment interface for a gastrointestinal sleeve or other device.

FIG. 11 illustrates an extra-lumen attachment cuff with an attachment interface for a gastrointestinal sleeve or other device.

FIG. 12 illustrates an intra-lumen attachment cuff with an attachment interface for a gastrointestinal sleeve or other device.

FIG. 13 illustrates an intra-lumen attachment cuff with an attachment interface for a gastrointestinal sleeve or other device.

FIGS. 14A-14D illustrate an intra-lumen attachment cuff with an attachment interface for a gastrointestinal sleeve or other device.

FIGS. 15A-15C illustrate various attachment options for the U or Y design attachment cuff of FIGS. 14A-14B using one or more T-tag fasteners.

FIG. 16 illustrates an attachment cuff or sleeve interfaced to the angle of the Z-line.

FIGS. 17A-17G illustrate a method of attaching an attachment cuff for a gastrointestinal sleeve or other device.

### DETAILED DESCRIPTION OF THE PREFERRED EMBODIMENT

Attachment cuff and methods of use for attachment of a gastrointestinal sleeve device or other implantable device have been described in the prior applications. The following represent novel embodiments of attachment cuffs. The examples given are not intended to be limiting. The various features and functions of the attachment cuffs described can be combined to create other embodiments as well. In the following description, the term “intra-lumen” attachment cuff will be used to describe an attachment cuff where the cuff is inside the gastric tissue such that gastric tissue is fastened around the exterior of the cuff only. The term “extra-lumen” attachment cuff will be used to describe an attachment cuff where the cuff is outside the gastric tissue such that at least a portion of the gastric tissue is fastened within the interior of the cuff, typically by plicating the gastric wall and attaching it within the cuff.

The prior applications describe the importance of minimizing the stress applied to the gastric walls to avoid premature dislodgement of the gastrointestinal sleeve device or other implantable device. Geometry of the device attachment and compliance of the attachment are among the strategies used to achieve this objective. Examples of structures using these strategies, which can be used separately or in addition and/or in combination with previously discussed strategies described herein and in the prior applications, are shown in the following figures.

In some clinical situations compliance requirements may be asymmetrical. For example attachment in a normally closed configuration at a normally closed sphincter such as the pylorus may require compliance in an outward direction but no inward compliance beyond the original attachment configuration. This can result in use of specific structure, for example use of a flexible elastic ring reinforced with wire may have little compliance inwardly or outwardly while use of a thread for reinforcement will allow inward motion while resisting outward motion, no reinforcement can allow motion in both directions.

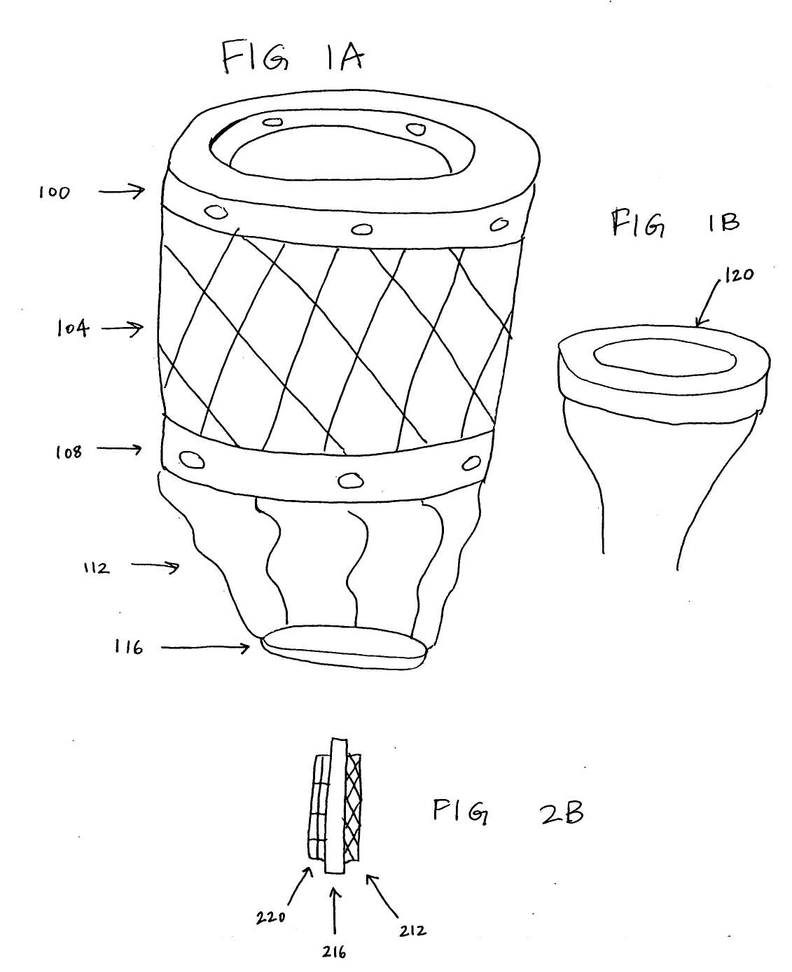

FIG. 1A illustrates an attachment device with specific functional zones delineated. At the top is a cylindrical zone with holes 100. Next is a crosshatched cylindrical zone 104 followed by a second cylindrical zone with holes 108. Next is an inverted conical (frustum) zone 112 transitioning from the second cylinder with holes and a rigid sleeve interface ring 116. FIG. 1B illustrates a sleeve with a rigid ring 120 at its proximal end, which is designed to interface with the rigid ring 116 at the distal end (bottom) of the attachment cylinder of FIG. 1A.

The cylindrical zones with holes, 100 and 108, are zones for attachment to the gastric wall. One or both of these zones can be used for this purpose. These zones are shown schematically and would be expected to have geometry and materials selected to optimize the interface with the gastric wall attachment means. The attachment zone of the cuff can be considered as part of the attachment means and for many clinical applications will preferably be constructed with a high compliance for a secure and long-lasting attachment to the gastric wall.

The crosshatched cylinder between these zones 104 is an area further optimized to interface with the gastric wall. For example materials in these zones can be optimized to encourage ingrowth, match or exceed the compliance of the gastric wall and/or redirect the forces associated with the attachment of the device to the gastric wall. This zone is also shown schematically and would be expected to have geometry and materials optimized for performance in these areas.

The inverted cone 112 is a schematic representation of the transition between the functions of gastric wall attachment and sleeve interface. Specifically, this zone can be structured to decouple forces related to one function from the other and minimize, for example, the negative effect of a rigid or low compliance sleeve interface relative to the gastric wall motion allowed by a highly compliant gastric wall interface. As a limiting example one can visualize a device with no such decoupling zone where a rigid sleeve interface directly attached to a compliant gastric wall interface would restrict the motion (compliance) of the gastric wall interface.

The sleeve interface ring 116 is also a schematic representation of any of a number of low compliance sleeve interface structures. Low compliance structure can be preferred in creating a leak free sleeve interface. Various sleeve interface configurations are described herein and in the prior applications.

In particular, these types of devices can allow the combination of a highly compliant gastric interface for robust gastric wall attachment with a secure leak free sleeve interface that is less compliant than would be desirable as a gastric wall interface.

Attachment: Attachment can be accomplished by means described herein and in the prior applications with attachment optimized for compliance providing for a maximum of gastric wall motion between attachment points with a minimum of resisting force.

Structures for compliant attachment to the stomach wall can be made in many manners.

1. stiff segments for attachment to the stomach with alternating stretchable or compliant segments;

2. pleats between attachment points (where the points can separate with little resistance until the pleats are fully straightened);

3. a compliant attachment ring attached to a compliant gastric wall interface structure (if applicable);

4. unconnected attachment points;

5. fenestrations (cuts or slits) between attachment points;

6. use of highly elastic compliant materials or structures (for example silicone or other polymers, knit or other fabrics or composites);

7. hinged, sliding, bellows and other structures.

Gastric wall interface: Materials for the gastric wall interface can be optimized for their lack of interaction with the gastric mucosa (e.g. silicone or fluoropolymers). Alternatively, these materials can be selected to encourage ingrowth and/or overgrowth (e.g. fabric, NiTi or other wire mesh or other materials described herein and in the prior applications.)

Stabilization of structures as well as improved mucosal overgrowth can be enhanced by increasing the porosity of ingrowth promoting materials with perforations or holes. This can be particularly helpful where one side of an implanted device (e.g. mounting cuff/ring) is in contact with gastric mucosa and the other side is exposed to gastric secretions.

Redirection of gastric wall forces and other means to avoid “cheese cutter” forces on attachment filaments have been discussed herein and in other applications. The attachment device of FIG. 1A can be used to explain how structural variations can optimize this type of performance.

For example, in the case of a device attached solely at the upper attachment cylinder, very little force redirection occurs since the gastric wall has minimal impingement with the device. In the case of the gastric wall attached to the OUTSIDE of the lower attachment cylinder, the gastric wall impinges along the tissue interface section of the attachment cuff and attachment forces can be thereby redirected. In the case of attachment to the INSIDE of the lower attachment cylinder the gastric wall or esophagus is further constrained and forced into a cylindrical configuration by the gastric interface section with which it is now coaxial. With attachment at the lower cylinder, attachment at the upper cylinder is optional and may be indicated in some clinical situations.

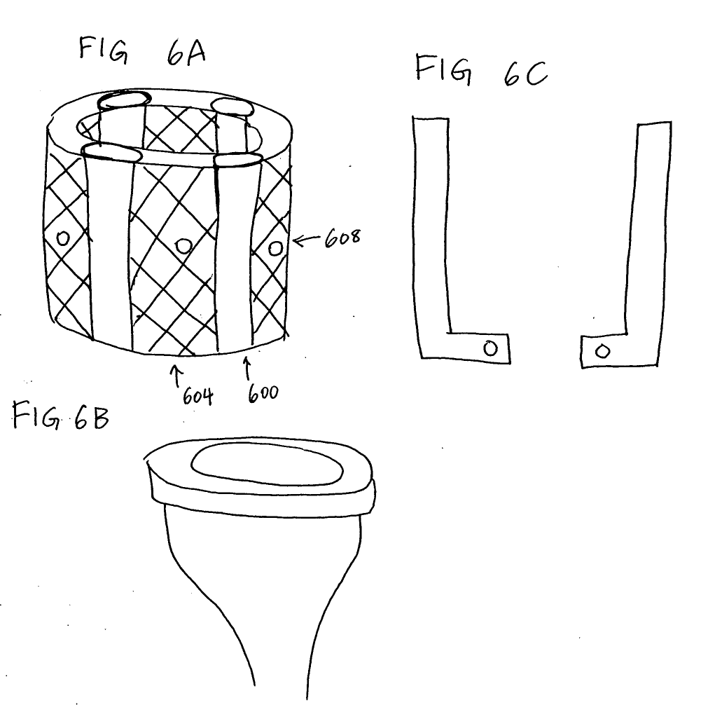

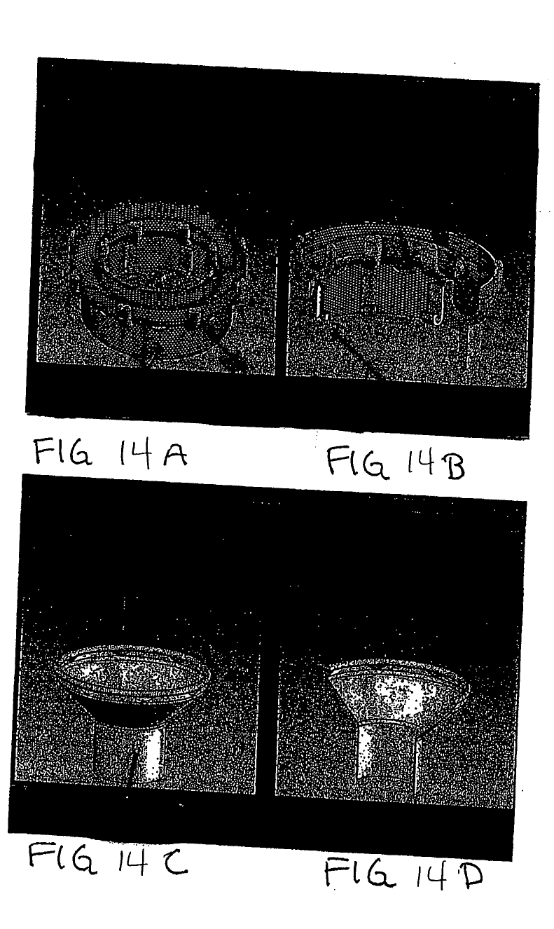

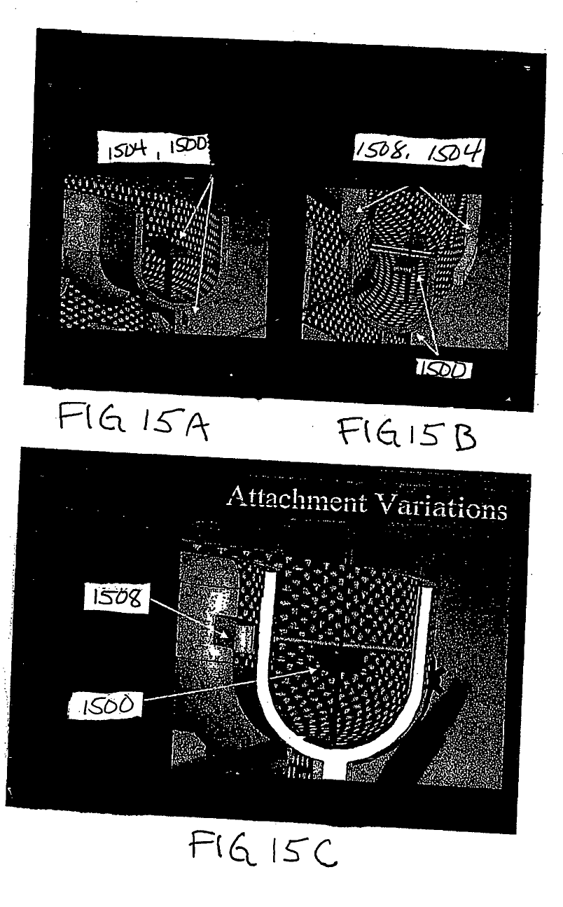

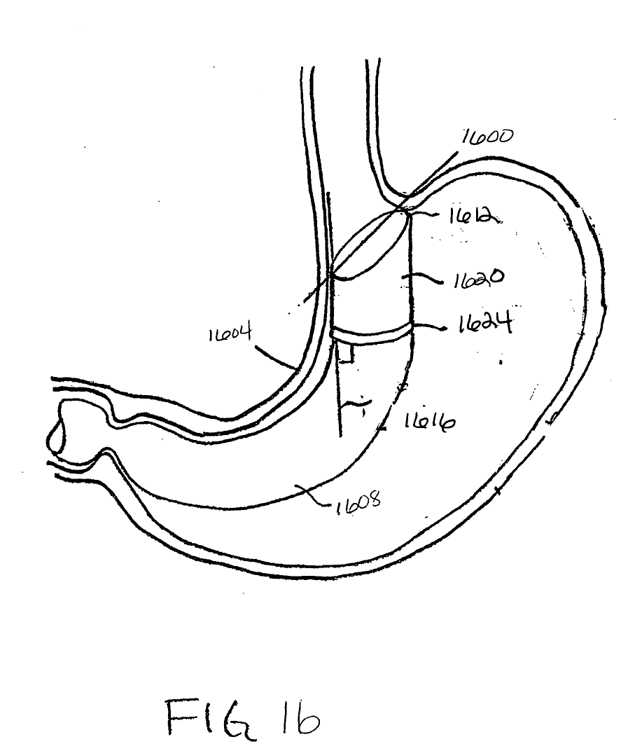

The structure of the device of FIG. 1A will influence the degree to which force redirection is effected. Rigidity or structure that resists collapse in the direction of the axis of the device will enhance force redirection. In the case of the gastric wall INSIDE the gastric interface section, resistance to radial stretch can also enhance force redirection. Radial rigidity can be balanced with radial compliance to optimize performance in these areas based upon clinical requirements. Structures that exhibit some or all of these principles include those depicted in FIGS. 6-14. The structure of FIG. 14 combined with the attachment shown in FIGS. 15B & 15C is particularly adapted to optimize force redirection.

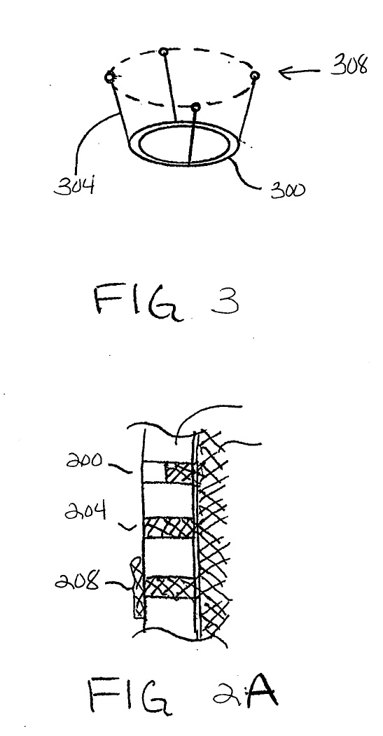

FIG. 2A shows ingrowth material with holes placed against the gastric mucosa. Various portions of FIG. 2A show:

1. Half way ingrowth 200;

2. Complete ingrowth 204;

3. Complete ingrowth+overgrowth+spreading 208.

FIG. 2B illustrates a tissue ingrowth mesh composite for use in the gastric wall interface zone of an attachment cuff or other implant device.

In this example the layers include:

1. Ingrowth layer 212 of mesh (Ti, NiTi or SS), fabric, expanded PTFE etc., can be impregnated with ingrowth encouraging chemicals (e.g. growth factors) etc.;

2. Barrier layer 216 to restrict cell migration (ingrowth) or wicking of a bonding agent into the ingrowth material;

3. Attachment layer 220 of fabric or other materials selected for bonding to other structures.

Decoupling transition: If a sleeve is attached directly to a compliant ring/cuff with no intermediate decoupling zone there will be limitations on the sleeve interface if it is desired to preserve the compliance of the ring/cuff. Specifically the sleeve-ring/cuff interface must be compliant. This can be accomplished:

1. with means/structures that match the structure and/or compliance of the ring/cuff;

2. mix and match sleeve and ring/cuff means/structures from the list above.

In addition to material and structures, geometry is a factor in decoupling. In general, the greater the difference in compliance of the structures, the longer the length of the decoupling zone. More highly compliant decoupling zone materials can mediate the requirement for increased length. Configurations which incorporate compliance (e.g. bellows or pleats) can also facilitate decoupling.

The structure shown in FIG. 3 illustrates an attachment device with a rigid or semi-rigid sleeve interface 300 attached to the gastric wall by way of flexible isolators 304 to provide attachment compliance. This is shown schematically in FIG. 3 as filaments attaching the sleeve interface 300 to the attachment points 308. This differs from FIG. 1A in that there is no gastric interface zone shown and the attachment points are shown isolated rather than being connected in a cylindrical configuration. Since it is desirable that there is little or no leakage of ingested food, the area encompassed by the filaments preferably includes some type of flexible compliant structure (e.g. a thin elastomeric film) shown by dashed lines between the flexible isolators 304. Many of the structures from the list above can be used as well to accomplish this result.

This can optionally be combined with a leak shield as described in the prior applications to facilitate a compliant but leak free attachment and interface. The leak shield can also be compliant. In the case of fenestrated structures, the leak shield can include overlapping slidable sections that can slide to accommodate motion at the attachment point while the overlap can accomplish a seal. Similarly, this structure can be used as a leak shield with a non-fenestrated device.

The following are exemplary embodiments of some of the concepts and structures described above and/or in other referenced documents.

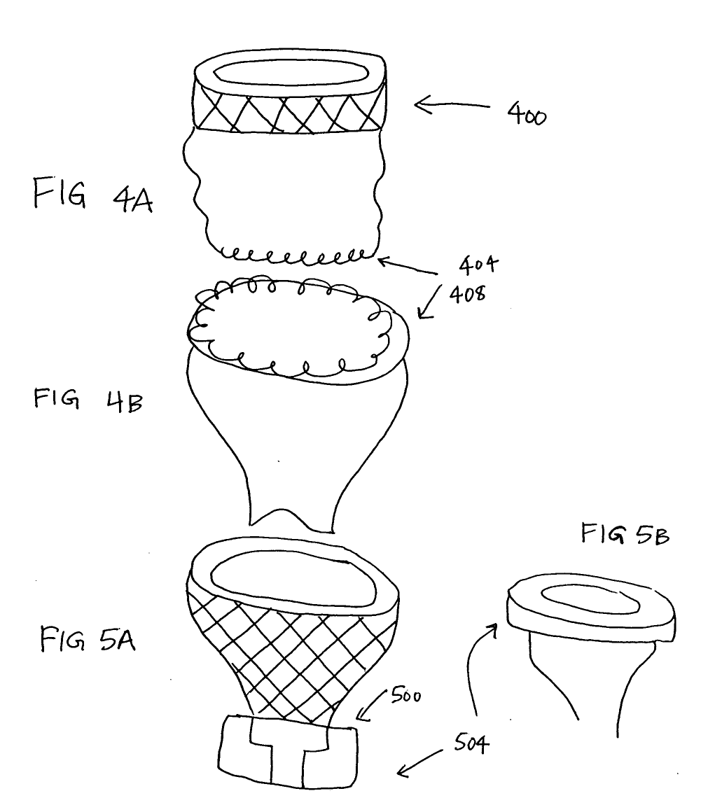

FIGS. 4A & 4B illustrate a basic intra-lumen attachment cuff with an attachment interface for a gastrointestinal sleeve or other device. The attachment cuffs attachment to the gastric wall is illustrated as a simple cylinder 400 of fabric and/or molded polymer. The sleeve interface 404 is shown as a NiTi spring coupling with a corresponding NiTi spring 408 on the sleeve. The attachment is decoupled from the sleeve attachment interface by a flexible and/or compliant segment between these two structures The length of this segment will vary depending upon the difference in compliance of the structures.

FIGS. 5A & 5B illustrate an alternate basic intra-lumen attachment cuff with an attachment interface for a gastrointestinal sleeve or other device. The sleeve attachment interface includes an inward-extending flange 500 that engages a corresponding outward-extending flange 504 on the proximal end of the gastrointestinal sleeve. The flanges may be made of, for example, overmolded silicone and can include wire, filamentous or other reinforcing. This device has a relatively rigid sleeve interface that is connected directly to the gastric interface material. This is an example of a device that does not include decoupling means/structure.

FIGS. 6A-6C illustrate an extra-lumen attachment cuff with an attachment interface for a gastrointestinal sleeve. The cuff has molded silicone compliant support columns 600 and is reinforced with a fabric and/or Ti or NiTi mesh 604 for resilient support. The silicone material is selected for compliance and would allow motion, stretch and compliance at the top, above and at other locations. Optionally, holes 608 may be provided in the mesh reinforced gastric interface sections for ease of attachment to the gastric wall. With appropriate material selection and geometry (distance) to the flange at the bottom, the gastric attachment and the sleeve interface can be effectively decoupled.

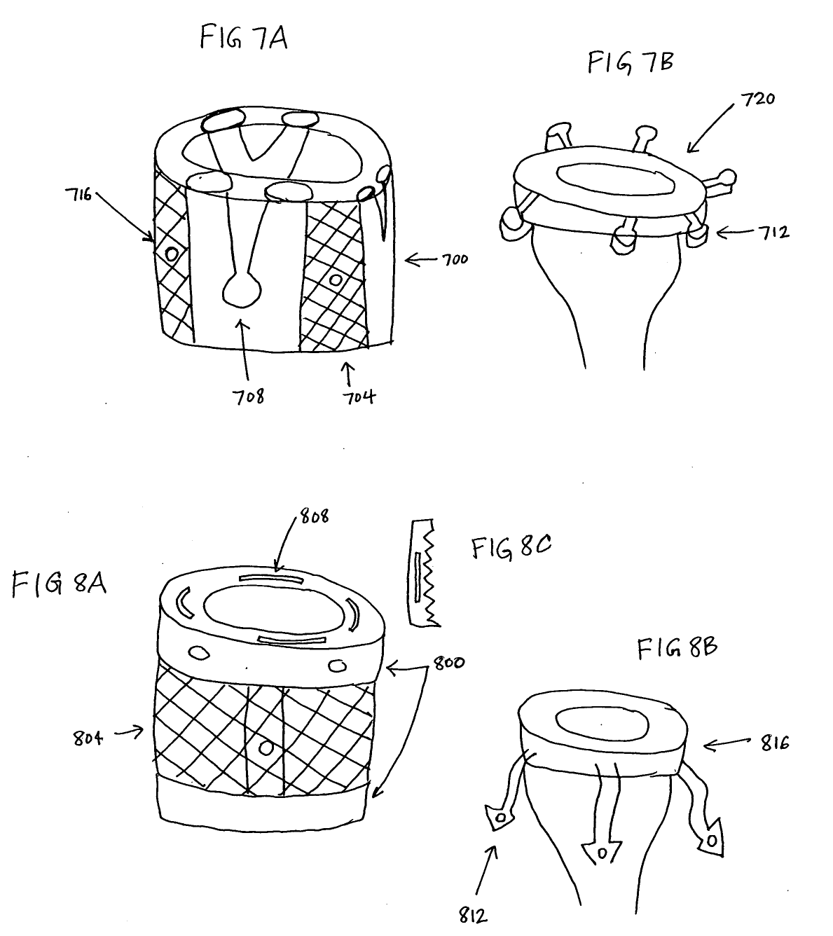

FIGS. 7A & 7B illustrate an intra-lumen attachment cuff with an attachment interface for a gastrointestinal sleeve or other device. The cuff has molded silicone support columns 700 and is reinforced with a fabric and/or Ti or NiTi mesh 704 for resilient support. The sleeve attachment interface includes tapered slots 708 in the support columns that engage a like number of keys 712 extending outwardly from a ring structure 720 on the proximal end of the gastrointestinal sleeve. The compliance of the gastric interface of this structure is greater that that of FIG. 6 since the slots 708 facilitate the relative motion of the attachment points on the reinforced gastric interface sections. Optionally, holes 716 may be provided in the mesh reinforced gastric interface sections 704 for ease of attachment to the gastric wall.

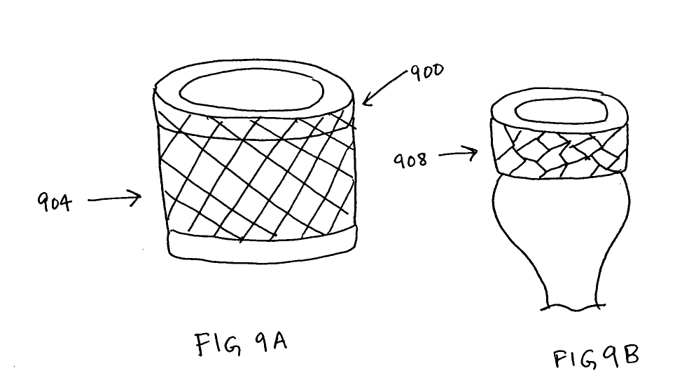

FIGS. 8A-8C illustrate an intra-lumen attachment cuff with an attachment interface for a gastrointestinal sleeve or other device on the top surface of the attachment cuff. The attachment cuff has molded silicone top and bottom rings 800 and a wall reinforced with a fabric and/or Ti or NiTi mesh 804 for resilient support. A cross-section of a molded ring 800 is illustrated in FIG. 8C. The sleeve attachment interface includes a plurality of slots 808 through the top ring of the attachment cuff that engage a like number of tabs 812 that extend downward from a ring structure 816 on the proximal end of the gastrointestinal sleeve. Preferably, the tabs 812 include a tapered detent or other locking mechanism to lock firmly into the slots 808.

FIGS. 9A-9B illustrate an intra-lumen attachment cuff and a gastrointestinal sleeve with an attachment interface for attaching to the cuff. The attachment cuff has molded silicone top and bottom rings 900 and a wall reinforced with a fabric, Ti or NiTi mesh 904 for resilient gastric interface/support. The selection of the resiliency and dimensions of the mesh can be selected to decouple the gastric attachment from the sleeve interface. The attachment interface includes a self-expanding (optionally braided) stent-like structure 908 on the proximal end of the gastrointestinal sleeve that expands to engage a reinforced (to prevent distension under the force of the expanding sleeve interface) interior surface of the attachment cuff.

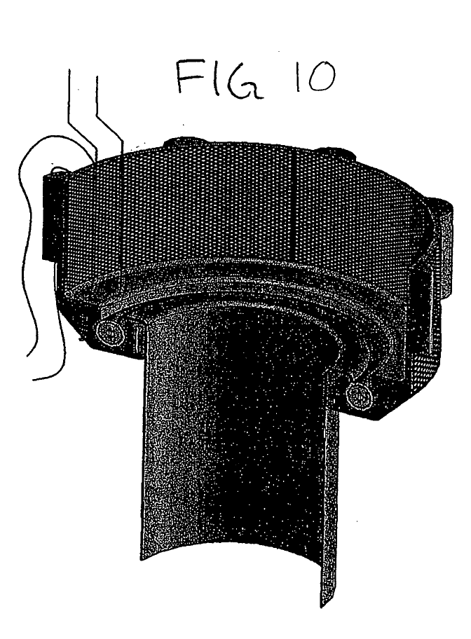

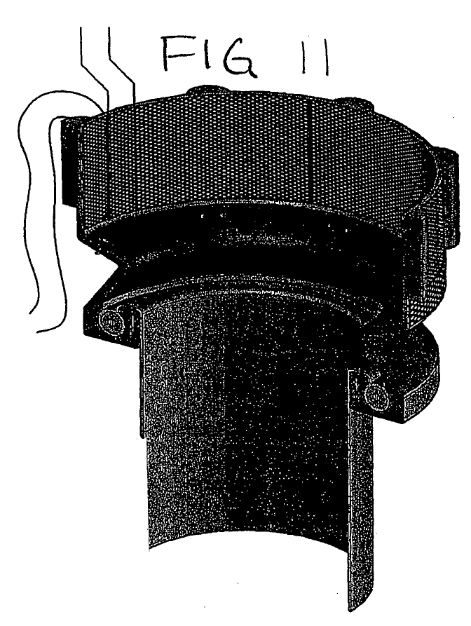

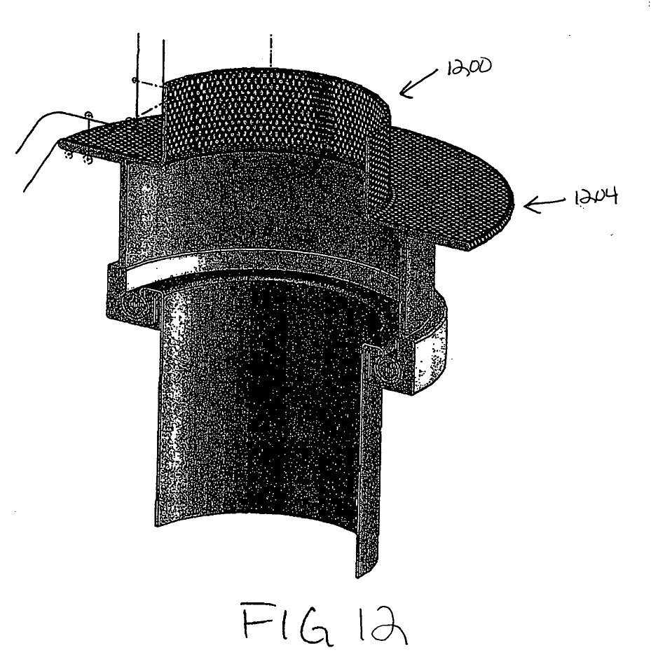

FIG. 10 illustrates an extra-lumen attachment cuff with an attachment interface for a gastrointestinal sleeve or other device. This embodiment of the device includes vertical molded supports and an improved flanged sleeve interface. Improvements in the sleeve interface include a cylindrical ribbon flange reinforcement (e.g. NiTi or PEEK, for reinforcing with increased compliance) and a flange trough with sleeve ring capturing. This example does not include decoupling means. The gastric wall is plicated and attached to the tissue attachment zone on the interior surface of the sleeve as shown on the left hand side of the drawing figure.

FIG. 11 illustrates an extra-lumen attachment cuff with an attachment interface for a gastrointestinal sleeve or other device. This is similar to FIG. 10, however this example includes a bellows decoupling structure between the gastric attachment and the sleeve interface. The gastric wall is plicated and attached to the tissue attachment zone on the interior surface of the sleeve as shown on the left hand side of the drawing figure.

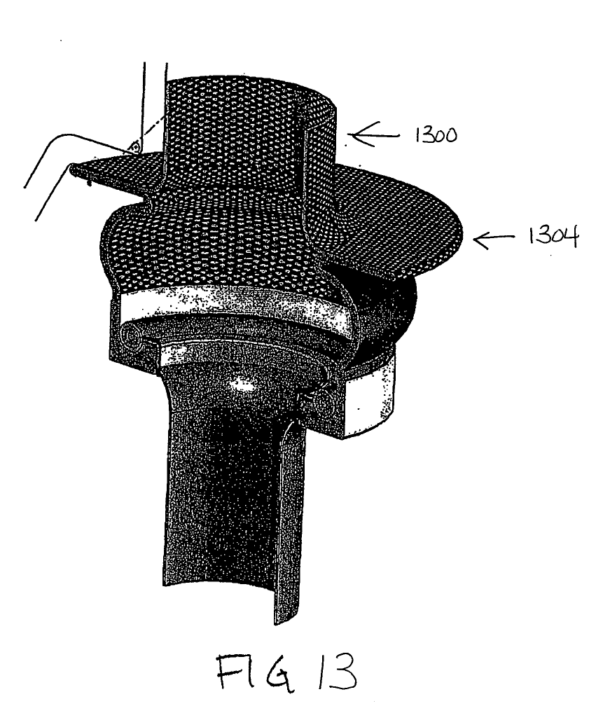

FIG. 12 illustrates an intra-lumen attachment cuff with an attachment interface for a gastrointestinal sleeve or other device. The attachment cuff includes a tissue attachment zone having an upward extending cylindrical wall 1200 with an annular flange 1204 at the bottom (Top Hat design) for attachment to the gastric wall. This example includes a cylindrical decoupling structure between the gastric attachment and the sleeve interface. The gastric wall is plicated and attached to the tissue attachment zone on the exterior surface of the sleeve as shown on the left hand side of the drawing figure.

FIG. 13 illustrates an intra-lumen attachment cuff with an attachment interface for a gastrointestinal sleeve or other device. Similar to FIG. 12, the attachment cuff includes a tissue attachment zone having an upward extending cylindrical wall 1300 with an annular flange 1304 at the bottom (Top Hat design) for attachment to the gastric wall. This example includes a bellows shaped decoupling structure between the gastric attachment and the sleeve interface. The gastric wall is plicated and attached to the tissue attachment zone on the exterior surface of the sleeve as shown on the left hand side of the drawing figure.

FIGS. 14A-14D illustrate an intra-lumen attachment cuff with an attachment interface for a gastrointestinal sleeve or other device. The attachment cuff includes a tissue attachment zone having inner and outer upward extending cylindrical walls or flanges at the top (U or Y design) for attachment to a plication in the gastric wall. This example includes a cylindrical decoupling structure between the gastric attachment and the hanger style sleeve interface. The attachment zone and/or tissue interface zone of the cuff is optionally made with velour or mesh material to encourage tissue ingrowth. The hanger style sleeve interface includes a multiplicity of molded hook-shaped sleeve hangers that are sewn or otherwise attached to the cuff material near the bottom of the decoupling zone. The proximal end of the sleeve is made with a reinforced rim having holes or cutouts to securely engage the hook-shaped sleeve hangers.

FIGS. 15A-15C illustrate various attachment options for the U or Y design attachment cuff of FIGS. 14A-14B using one or more T-tag fasteners.

Use of the plication T-tag 1500 of FIG. 15A can include structural features such as axial ribs to enhance force redirection. In the case of the use of dual T-tags 1504 in FIGS. 15B and 15C, the transverse T-tags 1508 kick the gastric wall and cuff device to further enhance force redirection. These attachment schemes combine the attributes of other methods and structures defined herein and in the prior applications. In addition to the force redirection mentioned herein, the plication T-tag 1500 acts as an extragastric buttress for the transverse T-tags 1508. Furthermore, these attachments can also be combined with other structures to control attachment tension and/or increase filament cross-sectional area.

Dual T-tags 1504 as shown in FIGS. 15A-15B are a variation of the anchors with fasteners discussed in prior applications where in this case a second T-tag is used to facilitate the fastening and securing of the primary T-tag anchor in place. The plication T-tag 1500 as shown in FIGS. 15A-15C is a variant of the use of an extragastric T-tag in that it can be used to help force or guide the gastric wall tissue into forming a plication, which has also been disclosed in prior applications. The use of the transverse T-tag 1508 as shown in FIGS. 15B-15C is another variation of fastening to a plication discussed in prior applications. The plication T-tags 1500 as shown in FIGS. 15B- 15C are a variation of use of a pledget T-tag as described in prior applications. In this case, the plication T-tag 1500 serves as the pledget T-tag and can be constructed as described in the prior applications.

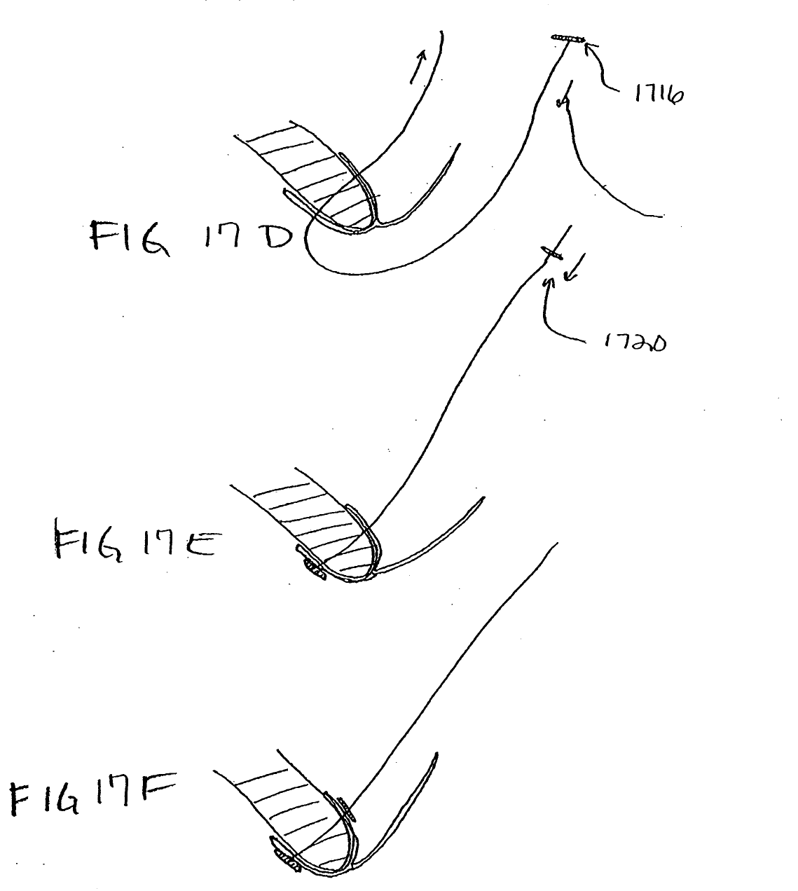

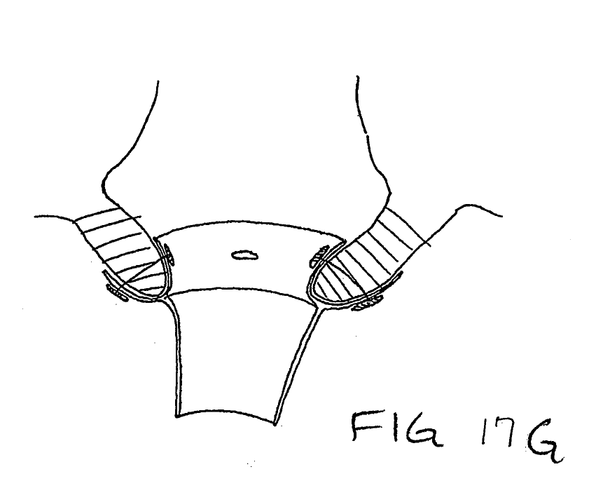

Methods applying the T-tags as shown in FIGS. 15B and 15C can be used as a primary procedure. The placement of the plication T-tags 1500 can be followed by parachuting a device into place and then attaching the transverse T-tags. This can be facilitated with a device to stabilize the plication and to simplify passage of the T-tag delivery device through the plication. This stabilization device can include a surgical or endoscopic forceps, which holds both sides of the plication while allowing means, e.g. holes in the arms of the forceps, for passage of the T-tag delivery device. Alternately, after or in conjunction with, placement of the plication T-tags 1500, the method outlined in FIGS. 17A-17G can be used to parachute the device into place.

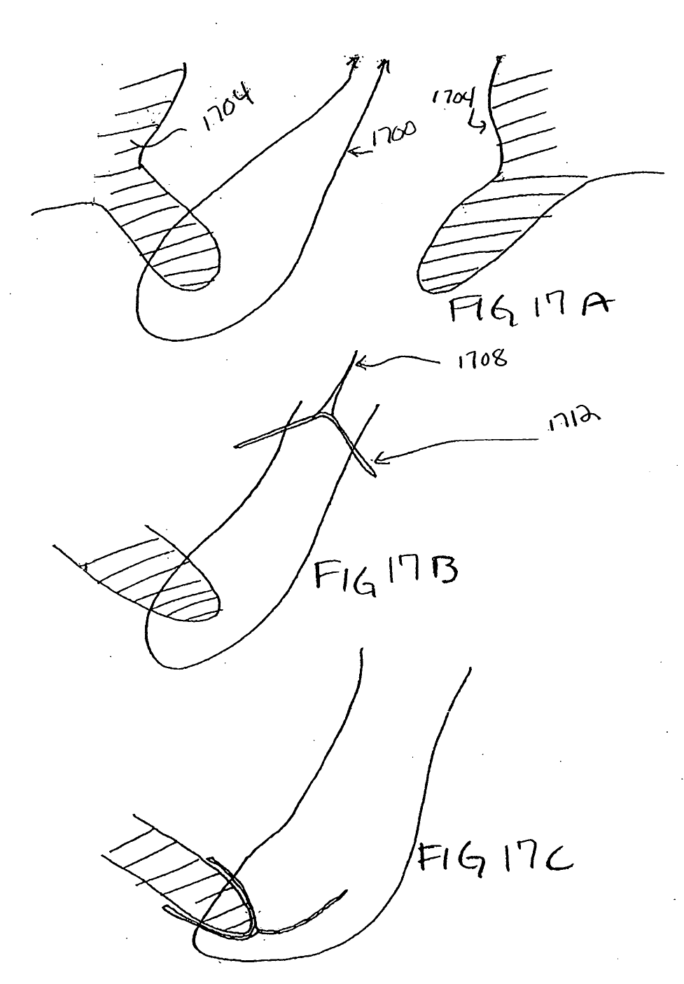

FIG. 16 illustrates an angled attachment cuff for attaching a gastrointestinal sleeve device at the Z-line 1600 or GEJ in a patient's digestive tract. As disclosed previously, it can be beneficial to distribute forces evenly around an attachment structure so forces on each attachment point will be equivalent and as low as possible. The stomach in humans (and pigs and dogs) curves from the GEJ to the pylorus. The Z-line 1600 or squamo-columnar mucosal junction is endoscopically visible and may or may not correspond to the GEJ which is the anatomic level at which the esophagus ends and the stomach begins. Forces along the axis of the sleeve 1608 would tend to force the sleeve 1608 against the lesser curve 1604. In this case forces transmitted to the sleeve attachment 1612 at the GEJ due to forces along the axis of the sleeve 1608 would be aligned with a line 1616 tangent to the lesser curve 1604. It can be preferable to attach structures at the GEJ or cardia of the stomach, as these are areas where tissue is thicker and stronger.

In FIG. 16 the sleeve is interfaced to the angle of the Z-line 1600 with an angled attachment cuff 1620. The angled attachment cuff 1620 allows attachment, for example, at the Z-line 1600 to be used with a sleeve interface 1624 at 90° to the axis of the sleeve 1608. This can be beneficial as it reduces or eliminates the need to orient an angled sleeve 1608 relative to the lesser curve 1604 of the stomach.

The following is an exemplary method for using an angled sleeve interface 1624. Some of the steps below could be redundant and could be skipped or combined. The order of the steps can be changed in some cases.

1. determine tissue interface/lesser curve related angle
assess anatomy
   swallow study
   CT
determine attachment points
   endoscopically identify Z-line
   place visual markers
   place RO markers to correlate with imaging
select appropriately angled devices
   angled cuff
   straight cuff/angled sleeve

2. place angled device
implant attachment fasteners, or -fasten device in place
Issues for step 2
attachment at previously identified attachment points orientation of angled device to obtain preferred orientation at and after placement

3. confirmation of orientation (optional)
radiological swallow study
ultrasound imaging

The prior art describes various sleeves for use in the GI tract. All describe an opening at the proximal end for food to enter the sleeve. Since many portions of the GI tract are potential channels, i.e. channels that are normally closed and open to allow passage of food, secretions, gases, etc., there may be situations where it is clinically desirable for the sleeve to be normally closed (like a lay flat tube) and be a potential channel.

Furthermore, if the proximal opening of the sleeve is secured to the GI tract at a sphincter (e.g. pylorus or LES) or portion of the GI tract that is a normally closed channel, it may be clinically desirable for the proximal end of the sleeve to also be normally closed. Additionally, it can be preferable for this proximal attachment to be configured as the tissue to which is attached. This puts minimal stress on the attachment while the attachment zone is at rest. This idea can be extended to the concept of having the attachment of the sleeve to move with the tissue to which it is attached with little or no (minimal) resistance, thereby minimizing stress on the attachment.

FIGS. 17A-17G illustrate a method of attaching an attachment cuff for a gastrointestinal sleeve or other device. This method can be particularly useful in parachute delivery of devices that capture a plication between two attachment-related structures as described herein and in the prior applications. The attachment cuff used in this method may be a Top Hat, U or Y design as described above.

Step 1 (FIG. 17A)—feed suture loop 1700 through a plication of the stomach wall 1704; hold on to both ends; repeat with multiple sutures around the inner periphery of the stomach

Step 2 (FIG. 17B)—feed one end of the suture through each side of the “Y” attachment 1712 on the superior side of the cuff 1708; repeat for additional sutures; parachute cuff down both ends of the suture loops at the same time

Step 3 (FIG. 17C)—the cuff is in place, except at this point the cuff is inverted in the superior direction

Step 4 (FIG. 17D)—add a knot or first anchor 1716 to the “far” side end of the suture loops; pull suture to seat first anchor

Step 5 (FIG. 17E)—add sliding second anchor 1720 to the other end of the suture loops and slide into position

Step 6 (FIG. 17F)—with the second anchor in position, give desired tissue compression then lock it and/or knot it in position; cut end of suture

Step 7 (FIG. 17G)—extend cuff distally

While the present invention has been described herein with respect to the exemplary embodiments and the best mode for practicing the invention, it will be apparent to one of ordinary skill in the art that many modifications, improvements and subcombinations of the various embodiments, adaptations and variations can be made to the invention without departing from the spirit and scope thereof.

What is claimed is:

1. An attachment device comprising
a flexible tubular cuff for attachment to tissue;
the cuff further comprising a plurality of holes for receiving T-tag anchors;
a gastrointestinal sleeve interface; and
a gastrointestinal sleeve;
wherein the sleeve is removably attached to the cuff at the sleeve interface; and
the sleeve comprises a plurality of holes for attachment to the cuff with fasteners.
